(19) United States
(12) Patent Application Publication
(10) Pub. No.: US 2015/0140684 A1
(43) Pub. Date: May 21, 2015

(54) TARGET AFFINITY MATERIAL INCLUDING BIODEGRADABLE POLYMER AND USE THEREOF

(71) Applicant: Samsung Electronics Co., Ltd., Suwon-si (KR)

(72) Inventors: Ga-hee KIM, Yongin-si (KR); Hyun-ju Kang, Hwaseong-si (KR); Ye-ryoung Yong, Seoul (KR); Jong-myeon Park, Incheon (KR)

(21) Appl. No.: 14/334,342

(22) Filed: Jul. 17, 2014

(30) Foreign Application Priority Data
Nov. 19, 2013 (KR) 10-2013-0140892

(51) Int. Cl. G01N 33/548; C08B 37/08

(52) U.S. Cl. CPC G01N 33/548; C08B 37/0072; G01N 2400/40
USPC 436/526; 530/391.1

(57) ABSTRACT

A target affinity material comprising a biodegradable polymer, wherein the biodegradable polymer comprises one or more solid particles and one or more materials that specifically binds to a target, as well as related methods and kits.

### RELATED APPLICATIONS

This application claims the benefit of Korean Patent Application No. 10-2013-0140892, filed on Nov. 19, 2013, in the Korean Intellectual Property Office, the disclosure of which is hereby incorporated by reference.

### BACKGROUND

1. Field

The present disclosure relates to a target affinity material including a biodegradable polymer and a use thereof.

2. Description of the Related Art

Diagnosis of a disease often requires separating a vesicle relevant (i.e. of interest or specific) to a particular disease (e.g., cancer) and analyzing a profile of a protein and a particular microRNA contained in the vesicle.

When the fluorescence-activated cell sorting (FACS) method is used to analyze a vesicle subpopulation in order to separate or detect a vesicle subpopulation, a sample may be distorted by cell loss, nonspecific binding, experimental complexity, and the sample needs to be prepared in advance. In addition, although a maximum of four antibodies may be used in a FACS experiment, the use of two or more antibodies decreases the experimental accuracy, and thus the type and number of antibodies which may be used are limited. In addition, the FACS method may decrease homogeneity of the results.

When an immunoaffinity bead is used to separate a vesicle and analyze a microRNA of the vesicle, a protein or a microRNA in the vesicle may be nonspecifically adsorbed to the bead when the vesicle is lysed, which may affect a subsequent experiment.

Therefore, it is necessary to inhibit nonspecific adsorption of a protein or a microRNA to an immunoaffinity bead in order to minimize the effect of the nonspecific adsorption on subsequent experiments.

### SUMMARY

Provided is a target affinity material including a biodegradable polymer, wherein the biodegradable polymer comprises one or more solid particles and one or more materials that specifically bind to a target.

Additionally, provided is a method of separating a target from a biological sample by using the target affinity material.

Furthermore, provided is a method of separating a target subpopulation from a biological sample by using the target affinity material.

### BRIEF DESCRIPTION OF THE DRAWINGS

These and/or other aspects will become apparent and more readily appreciated from the following description of the embodiments, taken in conjunction with the accompanying drawings in which:

FIG. 1A is a schematic diagram of an immunoaffinity material including a biodegradable polymer and a method of preparing the same;

FIG. 1B is a schematic diagram of a method of separating a target by using the immunoaffinity material;

FIG. 1C is a schematic diagram of a method of separating a target subpopulation by using the immunoaffinity material;

FIG. 2 is a graph displaying the result of an immunoblot performed using secondary antibodies to measure the degree of CD-9 introduction and the level of crosslinking of three types of immunoaffinity beads prepared according an embodiments of the present invention (1: Condition 1; 2: Condition 2; and 3: Condition 3);

FIG. 3 is a graph displaying the result of an immunoblot performed to measure and compare the capture efficiency of immunoaffinity beads and a magnetic bead coated with antibodies that were exposed to microvesicles;

FIG. 4A is a graph displaying the results of an immunoblot performed to measure and compare the non-specific adsorption of target microvesicles to different beads exposed to hyaluronic acid;

FIG. 4B is a graph displaying the results of an immunoblot showing the binding efficacy and non-specific adsorption of immunoaffinity beads and magnetic beads with and without exposure to hyaluronic acid; and

FIG. 5 is a graph displaying the results of a first microvesicle detection performed by using an immunoaffinity bead prepared according to an embodiment of the present invention (1 and 2) and the result of a second microvesicle detection performed after a hyaluronidase treatment (3 and 4).

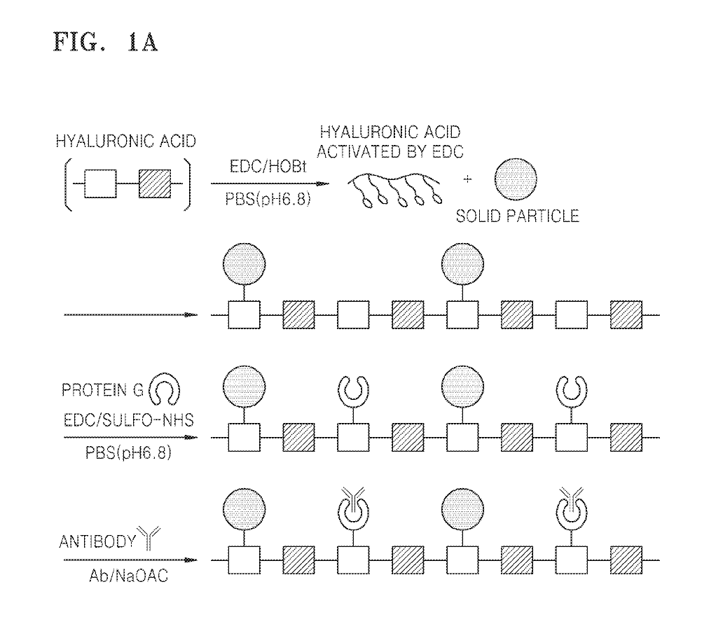

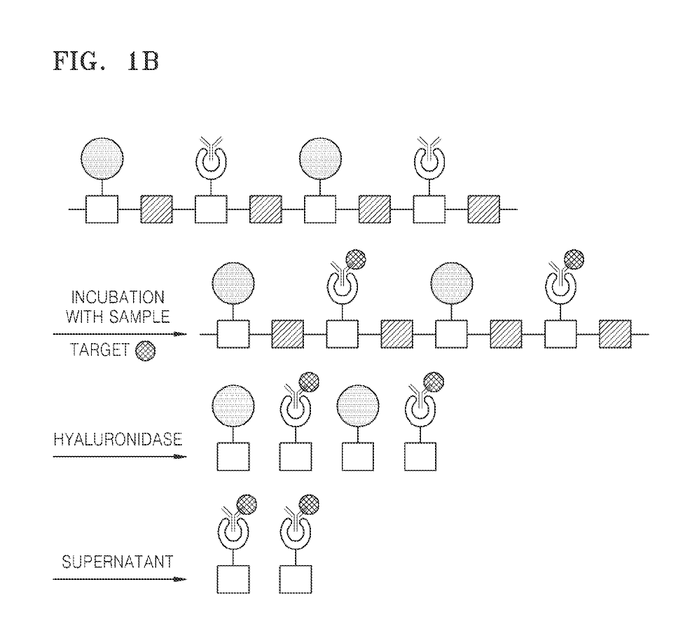

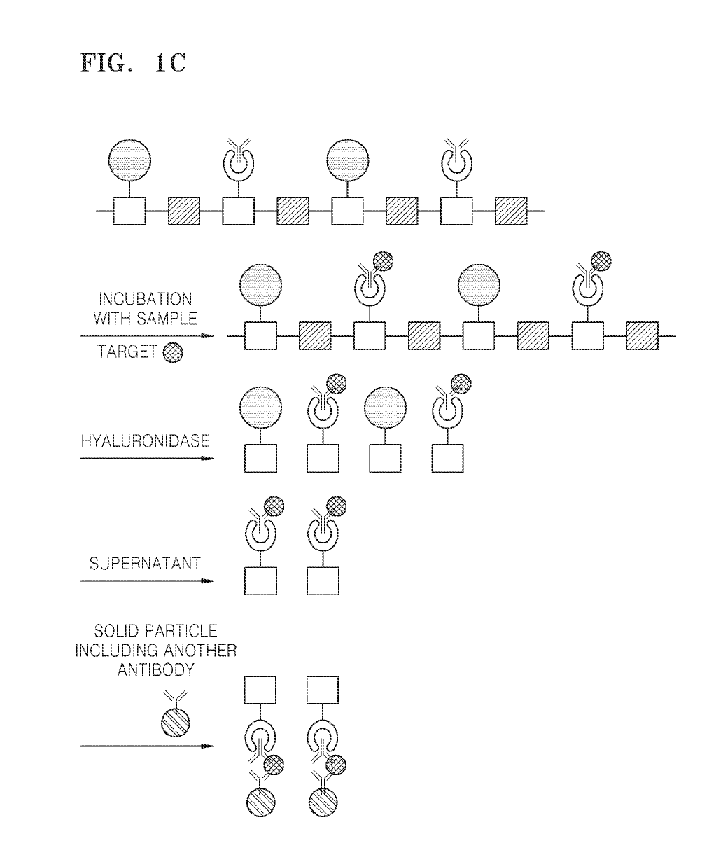

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items.

Provided is a target affinity material including a biodegradable polymer, wherein the biodegradable polymer comprises one or more solid particles and one or more materials that specifically bind to a target.

As used herein “target” refers to a material to be detected. The target may be, for example, a vesicle, a cell, a protein, a lipid, a sugar, or any combination thereof.

As used herein “vesicle” refers to a membrane structure surrounded by a lipid bilayer. For example, a vesicle may be a liposome or a microvesicle. The term “microvesicle” refers to a small vesicle having a cell-derived membrane structure. The term “microvesicle” is interchangeably used with the term “circulating microvesicle” or “microparticle.” The microvesicle may exist in a cell or be secreted from a cell. The microvesicle which is extracellularly secreted may comprise an exosome, an ectosome (also referred to as shedding microvesicle (SMV)), an apoptotic bleb, or any combination thereof. The exosome may be a phagocyte-derived membranous vesicle having a diameter from about 30 nm to about 100 nm. The ectosome may be a large-sized vesicle which is directly released from a plasma membrane, having a diameter from about 50 nm to about 1,000 nm. The apoptotic bleb may be a vesicle which is released from a dying cell, having a diameter from about 50 nm to about 3,000 nm. The microvesicle may in vivo include a microRNA or a messenger RNA (mRNA). A surface protein of a microvesicle may be a disease-specific marker.

A cell may be, for example, a cancer cell. A cancer may be cerebrospinal tumor, head and neck cancer, lung cancer, breast cancer, thymoma, mesothelioma, esophageal cancer, stomach cancer, colorectal cancer, liver cancer, pancreatic cancer, biliary tract cancer, renal cancer, bladder cancer, prostate cancer, testicular cancer, spermocytoma, ovarian cancer, cervical cancer, endometrial cancer, lymphoma, acute leukemia, chronic leukemia, multiple myeloma, sarcoma, malignant melanoma, skin cancer, or any combination thereof. A cell may be a circulating tumor cell (CTC) or a cancer stem cell (CSC).

A protein may be a surface protein of a vesicle or a cell. A lipid may be a membrane lipid of a vesicle or a cell. A sugar may be a sugar conjugated with a lipid or a surface protein existing on a vesicle or a cell membrane.

The term “material that specifically binds to a target” may be a ligand or other material that specifically binds to a protein, an enzyme substrate, a coenzyme, a regulatory factor, a material specifically bound to a receptor, a lectin, a sugar, a glycoprotein, an antigen, an antibody or an antigen-binding fragment thereof, a hormone, a neurotransmitter, a phospholipid-binding protein, a protein including a pleckstrin homology domain, a cholesterol-binding protein, or any combination thereof. The antigen-binding antibody fragment may include an antigen-binding site, for example, a single-domain antibody, Fab, Fab′, or scFv. The antibody or the antigen-binding fragment thereof may be bound to a biodegradable polymer via an immunoglobulin-binding protein. The immunoglobulin-binding protein may be Protein G, Protein A, Protein A/G, Protein L, or any combination thereof.

The solid particle may have a spherical shape, a polyhedral shape, a linear shape or any combination thereof. A solid particle may be a polystyrene particle, a polypropylene particle, a magnetic particle, or any combination thereof, but not limited thereto.

The biodegradable polymer may be a hydrophilic polymer. A hydrophilic polymer may decrease nonspecific binding by a protein.

The biodegradable polymer may include hyaluronic acid (HA), collagen, chitin, chitosan, heparin, or any combination thereof. A monomer of the biodegradable polymer may be hyaluronic acid (HA), collagen, chitin, chitosan, heparin, or any combination thereof. Hyaluronic acid is one of glycosaminoglycans and a polymer made of D-glucuronic acid and D-N-acetylglucosamine. Collagen is a protein which is a main component of extracellular matrix in animals and included in skin, tendon, cartilage, or others in a great quantity. Collagen has about 28 types. Chitin is a polysaccharide made of a amino-derivative of a sugar, which is formed by polymerization of N-acetylglucosamine by β-1,4 linkage. Chitosan is a material which is formed by deacetylating chitin so that chitin may be easily absorbed into a human body. Heparin is a glucosaminoglycan having a sulfate group in which most sulfate groups and carboxylate groups have a negative charge and heparin is widely used as an anticoagulant.

The biodegradable polymer may be degradable by an enzyme. The enzyme may be hyaluronidase, collagenase, chitinase, heparinase, or any combination thereof. Hyaluronidase is an enzyme hydrolyzing hyaluronic acid and a carbohydrase acting with a carbohydrate or a glycoside to hydrolyze a glycosidic bond. Hyaluronidase is also referred to as mucinase. Collagenase is a protein hydrolyzing enzyme degrading collagen. Collagenase hydrolyzes a bond between glycine and proline having a Pro-X-Gly-Pro-Y structure. Chitinase is an enzyme hydrolyzing a glycosidic bond of chitin. Heparinase refers to an endo-heparin lyase. The heparinase may be heparinase I, heparinase II, or heparinase III.

The one or more materials which may specifically bind to a target and the one or more solid particles may be bound to different sites of a biodegradable polymer. One or more materials may be bound to a biodegradable polymer and one or more solid particles may also be bound to a biodegradable polymer at the same time.

A target affinity material refers to a material which may be specifically bind to a target. A target affinity material including an antibody or an antigen-binding fragment thereof may also be referred to as an immunoaffinity material.

Provided is a method of separating a target from a biological sample including

incubating a biological sample including a target with a target affinity material to produce a complex of the target and the target affinity material in the biological sample, wherein the target affinity material includes a biodegradable polymer including one or more solid particles and one or more materials which specifically bind to the target;

separating the complex from the biological sample; and incubating the separated complex and a material that degrades the biodegradable polymer to separate the target from the complex.

The target, one or more materials that specifically bind the target, the solid particle, and the biodegradable polymer, are as described above.

The biological sample may be urine, mucus, saliva, blood, blood plasma, blood serum, sputum, spinal fluid, pleural fluid, nipple aspirate, lymph fluid, airway fluid, intestinal juice, genitourinary fluid, human milk, lymphoid body fluid, semen, cerebrospinal fluid, system body fluid, ascites, cystic tumor body fluid, amniotic fluid, or any combination thereof.

Incubating may be performed in vitro. Incubating may be performed, for example, in a temperature from about 0° C. to about room temperature. Incubating may be performed by performing, for example, rotation, vortexing, stirring, or any combination thereof.

Separating the complex from the biological sample may be performed by any methods known in this art. Separating may be performed by centrifugation, filtration, washing, dialysis, affinity chromatography, magnetic separation, density gradient method, free-flow electrophoresis, or any combination thereof.

The material that degrades the biodegradable polymer may be an enzyme. The enzyme may be hyaluronidase, collagenase, chitinase, heparinase, or any combination thereof. Hyaluronidase, collagenase, chitinase, and heparinase are as described above.

Separating the target from the complex may further include other isolation methods known in this art. Separating may include, for instance, centrifugation, filtration, washing, dialysis, affinity chromatography, magnetic separation, density gradient method, free-flow electrophoresis, or any combination thereof.

The method may further include detecting the separated target. Detection may be performed by any methods known in this art. Detection may be performed by, for example, immunoblotting, immunoprecipitation, chromatography, mass spectrometry, protein array, polymerase chain reaction (PCR), reverse transcription-polymerase chain reaction (RT-PCR), microarray, or any combination thereof.

Provided is a method of separating a target subpopulation from a biological sample including

incubating a biological sample including a target with a target affinity material to produce a complex of the target and the target affinity material in the biological sample, wherein the target affinity material includes a biodegradable polymer including one or more solid particles and one or more first materials that specifically bind the target;

separating the complex from the biological sample;

incubating the separated complex and a material that degrades the biodegradable polymer to separate the target from the complex; and

incubating to the target with a second material that specifically binds the target (or a subpopulation of the target) to separate a target subpopulation, which is specifically bound to the second material, wherein the second material is different from the first material;

and, optionally, repeating the incubation of the target subpopulation with one or more additional materials that specifically bind the same or a different target subpopulation and separating another target subpopulation.

The target affinity material may be a biodegradable polymer including one or more first materials that specifically bind to the target and one or more solid particles. The target, the target affinity material which may specifically bind to the target, the solid particle, the biodegradable polymer, and the complex are as described above.

The material degrading the biodegradable polymer, incubating the separated complex and the material degrading the biodegradable polymer, and separating thereof are as described above.

Incubating the target with one or more second materials that specifically bind to the target and separating the target subpopulation which is specifically bound to the second material are as described above.

The first material and the second material may specifically bind to different sites of a target. For example, the first material and the second material may be different antibodies which specifically bind to different antigens or different epitopes.

The method may further include detecting the separated target subpopulation. Detection is as described above.

Provided is a composition or a kit including a target affinity material including a biodegradable polymer including one or more materials that specifically bind to a target or one or more solid particles for separating one or more targets.

The target, the target affinity material which may specifically bind to the target, the solid particle, and the biodegradable polymer are as described above.

The composition or kit may further include an enzyme which may degrade a biodegradable polymer. The enzyme may be, for example, hyaluronidase, collagenase, chitinase, heparinase, or any combination thereof. Hyaluronidase, collagenase, chitinase, and heparinase are as described above.
Provided is a method of preparing a target affinity material including activating a biodegradable polymer;
combining the activated biodegradable polymer with one or more solid particles to bind the biodegradable polymer to the one or more solid particles; and
combining the biodegradable polymer bound to one or more solid particles with one or more materials that specifically bind to a target.

The biodegradable polymer, the solid particle, and the material that specifically binds to a target are as described above.

Activating a biodegradable polymer may be performed by combining a functional group of the biodegradable polymer with a coupling reagent. The term “functional group” refers to specific groups of atoms or bonds within molecules that are responsible for the characteristic chemical reactions of those molecules. Examples of functional groups are known to those of ordinary skill in the art.

The coupling reaction may be performed by using a coupling reagent. The coupling reagent may be, for example, 1-ethyl-3-(3-dimethylaminopropyl) carbodiimide (EDC) or 1-hydroxybenzotriazole (HOBt).

The solid particle may include a functional group that binds the activated biodegradable polymer. The functional group may be, for example, an amine group.

When the material that specifically binds the target is an antibody, combining the biodegradable polymer bound to one or more solid particles with one or more materials that specifically bind the target may further include combining the biodegradable polymer bound to one or more solid particles with a immunoglobulin-binding protein so that the protein is bound to the polymer; and combining the biodegradable polymer bound to the one or more solid particles and the immunoglobulin-binding protein with a material that specifically binds to the target. The immunoglobulin-binding protein is as described above.

Hereinafter, the present invention will be described in further detail with reference to examples. It will be obvious to a person having ordinary skill in the art that these examples are illustrative purposes only and are not to be construed to limit the scope of the present invention.

### Example 1

### Preparation of Immunoaffinity Bead to which Hyaluronic Acid and Antibody are Introduced

An immunoaffinity bead was prepared under several conditions and the degree of antibody introduction was compared to verify the level of the immunoaffinity bead preparation.

10 mg/ml of hyaluronic acid (Sigma), 25.4 mg/ml of 1-ethyl-3-(3-dimethylaminopropyl) carbodiimide (EDC) (Sigma), 17.9 mg/ml of 1-hydroxybenzotriazole (HOBt) (Sigma), and 1 ml of phosphate buffered saline (PBS) (pH 6.8) were mixed and the resulting mixture was incubated at 4° C. for 12 hours to activate hyaluronic acid.

Under Condition 1, 48 μl of activated hyaluronic acid and 100 μl of DYNABEADS® M-270 Amine (Invitrogen) were mixed and the resulting mixture was incubated at room temperature for one hour. Then, 3 μl of Protein (Sigma), 75 mg/ml of EDC (Sigma), 75 mg/ml of sulfo-N-hydroxysulfosuccinimide (sulfo-NHS) (Sigma), and 500 μl of PBS (pH 6.8) were mixed and the resulting mixture was incubated at room temperature for one hour. The reaction product was mixed with 160 μl of an anti-CD9 antibody (500 mg/ml) (R&D Systems) and the resulting mixture was incubated at room temperature for three hours.

Under Condition 2, 48 μl of activated hyaluronic acid and 3 μl of Protein G (10 mg/ml) (Sigma) were mixed and the resulting mixture was incubated at room temperature for one hour. Then, 100 μl of DYNABEADS® M-270 Amine (Invitrogen), 75 mg/ml of EDC (Sigma), 75 mg/ml of sulfo-NHS (Sigma), and 500 μl of PBS (pH 6.8) were mixed and the resulting mixture was incubated at room temperature for one hour. The reaction product was mixed with 160 μl of an anti-CD9 antibody (500 mg/ml) (R&D Systems) and the resulting mixture was incubated at room temperature for three hours.

Under Condition 3, 48 μl of activated hyaluronic acid, 3 μl of Protein G (10 mg/ml) (Sigma), and 100 μl of DYNABEADS® M-270 Amine (Invitrogen) were mixed and the resulting mixture was incubated at room temperature for three hours. The reaction product was mixed with 160 μl of an anti-CD9 antibody (500 mg/ml) (R&D Systems) and the resulting mixture was incubated at room temperature for three hours.

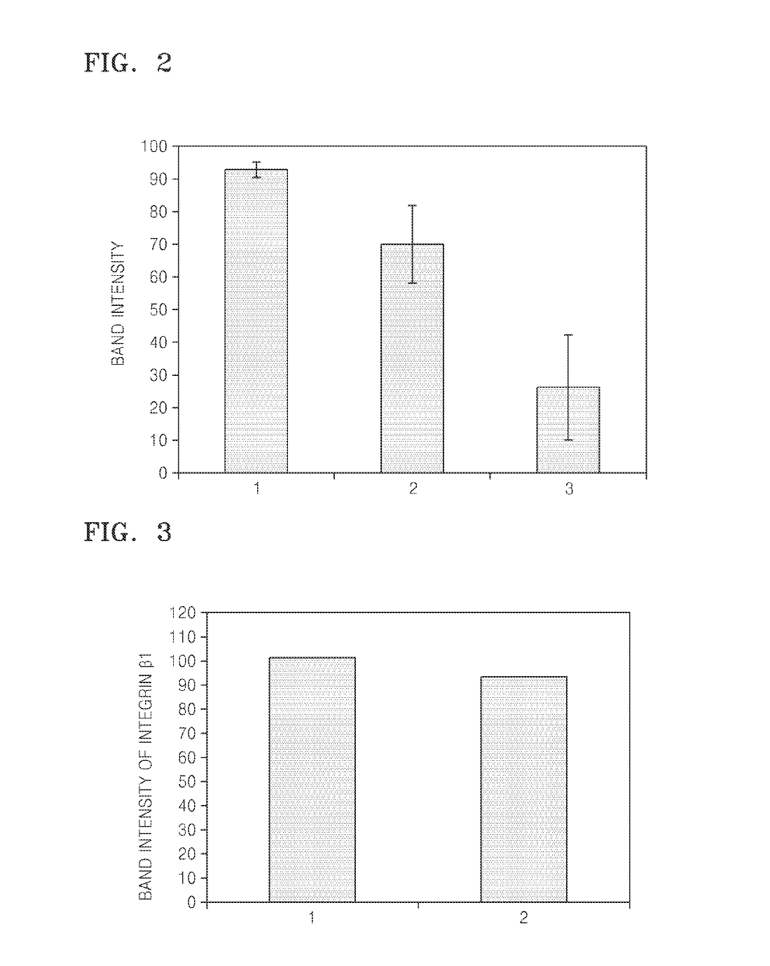

To verify the level of anti-CD9 antibody introduction and the level of cross-linking, an immunoblotting was performed with the prepared immunoaffinity bead by using a secondary antibody with florescence material (BioRad). FIG. 2 shows the quantified results of the obtained band intensity (1: Condition 1; 2: Condition 2; and 3: Condition 3). In addition, Table 1 shows the immunoaffinity bead preparation conditions and the corresponding immunoaffinity bead preparation levels.

TABLE 1

| | | Immunoaffinity |
|---|---|---|
| | | Bead |
| Preparation Condition | | Preparation Level |
| Condition | (activated hyaluronic acid + amine bead), | High |
| 1 | and then Protein G | |
| Condition | (activated hyaluronic acid + Protein G), | Medium |
| 2 | and then amine bead | |
| Condition | activated hyaluronic acid + amine bead + | Low |
| 3 | Protein G | |

As shown in FIG. 2 and Table 1, it was verified that, by the preparation method of combining activated hyaluronic acid and amine bead and then combining Protein G with the reaction product, the antibody was well introduced.

### Example 2

### Detecting of Microvesicle in Blood by Using Immunoaffinity Bead to which Activated Hyaluronic Acid and Antibody are Introduced

The immunoaffinity bead to which the hyaluronic acid and the anti-CD9 antibody were introduced, which was prepared by the method of Condition 1 described in Example 1, was used to detect a microvesicle in blood and a capturing efficiency was compared.

A blood sample of a healthy person was taken and centrifuged to obtain a blood plasma.

0.3 ml of the blood plasma and 30 μl of the immunoaffinity bead to which the hyaluronic acid and the anti-CD9 antibody were introduced were mixed and the resulting mixture was incubated at room temperature for four hours. The reaction product was washed with PBS and a microvesicle was separated.

To detect a microvesicle by a conventional method, a magnetic bead (Invitrogen) was coated with an anti-CD9 antibody (R&D Systems). 0.3 ml of the blood plasma and the magnetic bead coated with an anti-CD9 antibody were mixed and the resulting mixture was incubated at room temperature for four hours. The reaction product was washed with PBS and a microvesicle was separated.

The separated microvesicle, an LDX sample buffer (Sigma), and a reducing-agent (Invitrogen) were mixed and the resulting mixture was incubated at room temperature to denature and reduce a protein included in the microvesicle. An immunoblotting was performed with the lysed microvesicle by using an anti-integrin β1 antibody (Abcam). The band intensity of the obtained integrin β1 is shown in FIG. 3 (1: immunoaffinity bead to which hyaluronic acid and an anti-CD9 antibody were introduced; and 2: magnetic bead coated with an anti-CD9 antibody).

As shown in FIG. 3, it was verified that the microvesicle capture efficiency of the immunoaffinity bead to which the hyaluronic acid and the anti-CD9 antibody were introduced was similar to the microvesicle capture efficiency of the magnetic bead coated with an antibody.

### Example 3

### Detecting of Microvesicle by Nonspecific Adsorption of Bead to which Hyaluronic Acid is Introduced

It was verified whether a bead which did not include an antibody and to which hyaluronic acid was introduced was nonspecifically adsorbed to a microvesicle.

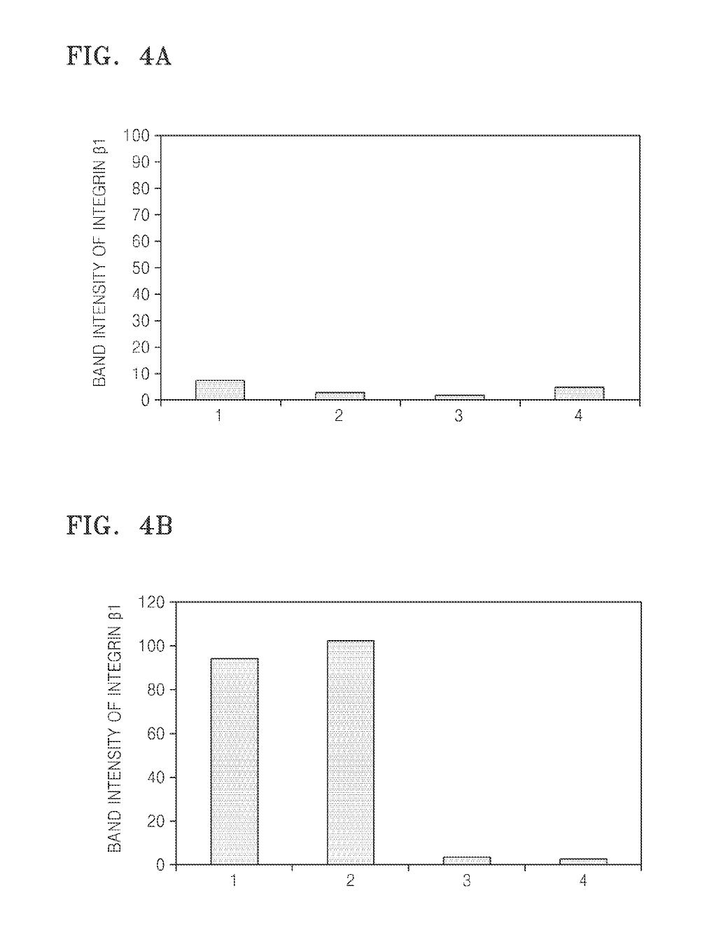

48 μl of activated hyaluronic acid which was prepared by the method described in Example 1 was mixed with 100 μl of DYNABEADS® M-270 Amine (Invitrogen) and the resulting mixture was incubated at room temperature for one hour. The prepared bead was washed with PBS. 30 μl of the washed bead was mixed with 0.3 ml of blood plasma and the resulting mixture was incubated at room temperature for four hours to bind the bead to which the hyaluronic acid was introduced and a microvesicle in the blood plasma. The reaction product was washed with PBS, and then, an LDX sample buffer (Sigma) and a reducing-agent (Invitrogen) were added into the reaction product. An immunoblotting was performed with the reaction product by using an anti-integrin β1 antibody (Abcam). The band intensity of the obtained integrin β1 is shown in FIGS. 4a (1 to 4: beads which did not include an antibody and to which hyaluronic acid was introduced).

As shown in FIG. 4a, almost no integrin β1, which is a marker of a microvesicle, was detected. Therefore, it was verified that a microvesicle was not nonspecifically adsorbed to the bead to which hyaluronic acid was introduced.

These results were compared to an immunoaffinity bead to which the hyaluronic acid and the anti-CD9 antibody were introduced, which was prepared by the method of Condition 1 described in Example 1, and a magnetic bead coated with the anti-CD9 antibody which was prepared by the method described in Example 2 was prepared. The prepared bead was mixed with 30 μl of blood plasma and the resulting mixture was incubated at room temperature for four hours. To 10 μl of the reaction product, an LDX sample buffer (Sigma) and a reducing-agent (Invitrogen) were added. An immunoblotting was performed with the resulting mixture by using an anti-integrin β1 antibody (Abcam) to perform a first detection of a microvesicle in the blood plasma. 10 μl of the reaction product was not treated with hyaluronidase and the bead in the reaction product was separated by using a magnet. An immunoblotting was performed with a remaining supernatant by using an anti-integrin β1 antibody (Abcam) to perform a second detection of a microvesicle in the blood plasma. The band intensity of the obtained integrin β1 is shown in FIG. 4b (1: the first detection by using the magnetic bead coated with an anti-CD9 antibody; 2: the first detection by using the immunoaffinity bead to which hyaluronic acid and an anti-CD9 antibody were introduced; 3: the second detection by using the magnetic bead coated with an anti-CD9 antibody without treating the reaction product with hyaluronidase; and 4: the second detection by using the immunoaffinity bead to which hyaluronic acid and an anti-CD9 antibody were introduced without treating the reaction product with hyaluronidase).

As shown in FIG. 4b, in the case where a microvesicle was captured by using the immunoaffinity bead or the magnetic bead and the reaction product was not treated with hyaluronidase, no microvesicle was detected in the supernatant. Therefore, it was verified that the detected band obtained by treating the reaction product with hyaluronidase was not caused from nonspecific binding of a microvesicle to an immunoaffinity bead.

### Example 4

### First Microvesicle Detection by Using Immunoaffinity Bead to which Hyaluronic Acid was Introduced and Second Microvesicle Detection after Hyaluronidase Treatment

An immunoaffinity bead to which the hyaluronic acid and the anti-CD9 antibody were introduced, which was prepared by the method of Condition 1 described in Example 1, and a magnetic bead coated with an anti-CD9 antibody which was prepared by the method described in Example 2 was prepared.

30 μl of the immunoaffinity bead to which the hyaluronic acid and an anti-CD9 antibody were introduced was mixed with 0.3 ml of blood plasma and the resulting mixture was incubated at room temperature for four hours. The reaction product was washed with PBS to separate a microvesicle. To the reaction product including the separated microvesicle, an LDX sample buffer (Sigma) and a reducing-agent (Invitrogen) was added. An immunoblotting was performed with the reaction product by using an anti-integrin β1 antibody (Abcam) to perform a first detection of a microvesicle in the blood plasma.

300 μl of the reaction product including the separated microvesicle and 100 units of hyaluronidase (Sigma) were mixed and the resulting mixture was incubated at room temperature for 30 minutes. The reaction product and the magnetic bead coated with an anti-CD9 antibody were mixed and the resulting mixture was incubated at room temperature for four hours. To the reaction product, an LDX sample buffer (Sigma) and a reducing-agent (Invitrogen) were added. An immunoblotting was performed with the reaction product by using an anti-integrin β1 antibody (Abcam) to perform a second detection of a microvesicle.

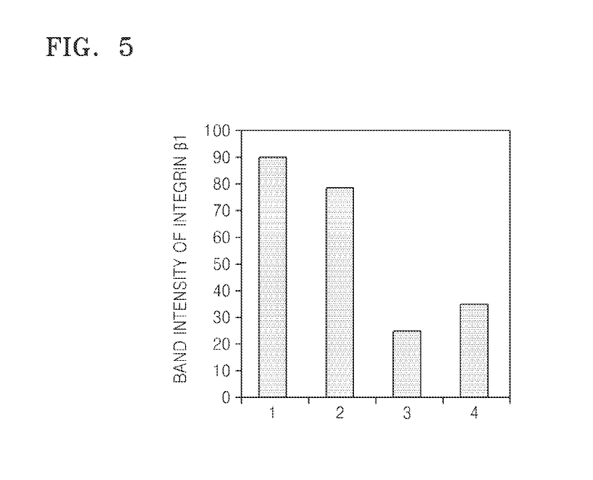

The results of the first and the second microvesicle detection are shown in FIGS. 5 (1 and 2: first microvesicle detection by using the immunoaffinity bead to which an anti-CD9 antibody and hyaluronic acid were introduced; and 3 and 4: second microvesicle detection by using a magnetic bead which was first treated with hyaluronidase and then an anti-CD9 antibody was introduced).

As shown in FIG. 5, the microvesicle detected by using the immunoaffinity bead to which hyaluronic acid was introduced was re-detected after a hyaluronidase treatment.

As described above, according to a target-affinity material and a use thereof according to an embodiment of the present invention, the effect of nonspecific adsorption is minimized, and an intact target may be directly separated or detected from a biological sample and a subpopulation of the target may be separated or detected. In addition, as a same sample may be repeatedly used, highly accurate analytical results may be obtained.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A target affinity material comprising a biodegradable polymer, wherein the biodegradable polymer comprises one or more solid particles and one or more materials that specifically binds to a target.

2. The target affinity material according to claim 1, wherein the one or more materials that specifically bind to a target and the one or more solid particles are bound to different sites of the biodegradable polymer.

3. The target affinity material according to claim 1, wherein the one or more materials that specifically bind to a target are each, independently, a ligand or other material that bind to a protein, an enzyme substrate, a coenzyme, a regulatory factor, a receptor, a lectin, a sugar, a glycoprotein, an antigen, an antibody or an antigen-binding fragment thereof, a hormone, a neurotransmitter, a phospholipid-binding protein, a protein including a pleckstrin homology domain, a cholesterol-binding protein, or any combination thereof.

4. The target affinity material according to claim 1, wherein the one or more materials that specifically bind a target include an antibody or antigen-binding antibody fragment, and the antibody or the antigen-binding fragment is bound to the biodegradable polymer via an immunoglobulin-binding protein.

5. The target affinity material according to claim 1, wherein the one or more solid particles is a polystyrene particle, a polypropylene particle, a magnetic particle, or any combination thereof.

6. The target affinity material according to claim 1, wherein the biodegradable polymer is a hydrophilic polymer.

7. The target affinity material according to claim 1, wherein the biodegradable polymer comprises hyaluronic acid, collagen, chitin, chitosan, heparin, or any combination thereof.

8. The target affinity material according to claim 1, wherein the biodegradable polymer is an enzyme-degradable polymer.

9. The target affinity material according to claim 8, wherein the enzyme-degradable polymer is a polymer degradable by hyaluronidase, collagenase, chitinase, heparinase, or any combination thereof.

10. A method of separating a target from a biological sample comprising
incubating a biological sample comprising a target with a target affinity material of claim 1 to produce a complex of the target and the target affinity material in the biological sample;
separating the complex from the biological sample; and
incubating the separated complex with a material that degrades the biodegradable polymer to separate the target from the complex.

11. The method according to claim 10, wherein the biological sample comprises urine, mucus, saliva, blood, blood plasma, blood serum, sputum, spinal fluid, pleural fluid, nipple aspirate, lymph fluid, airway fluid, intestinal juice, genitourinary fluid, human milk, lymphoid body fluid, semen, cerebrospinal fluid, system body fluid, ascites, cystic tumor body fluid, amniotic fluid, or any combination thereof.

12. The method according to claim 10, wherein the target comprises a vesicle, a cell, a protein, a lipid, a sugar, or any combination thereof.

13. The method according to claim 10, wherein the one or more materials that specifically bind a target comprise a ligand or other material that specifically binds a protein, an enzyme substrate, a coenzyme, a regulatory factor, a receptor, a lectin, a sugar, a glycoprotein, an antigen, an antibody or an antigen-binding fragment thereof, a hormone, a neurotransmitter, a phospholipid-binding protein, a protein including a pleckstrin homology domain, a cholesterol-binding protein, or any combination thereof.

14. The method according to claim 10, wherein the material that degrades the biodegradable polymer is an enzyme.

15. The method according to claim 14, wherein the enzyme is hyaluronidase, collagenase, chitinase, heparinase, or any combination thereof.

16. The method according to claim 10, wherein separating the complex from the biological sample, separating the target from the complex, or both comprises centrifugation, filtration, washing, dialysis, affinity chromatography, magnetic separation, density gradient method, free-flow electrophoresis, or any combination thereof.

17. The method according to claim 10, further comprising detecting the separated target.

18. A method of separating a target subpopulation from a biological sample comprising:
incubating a biological sample comprising a target and a target affinity material according to claim 1 to produce a complex of the target and the target affinity material in the biological sample;
separating the complex from the biological sample;
incubating the separated complex with a material that degrades the biodegradable polymer to separate the target from the complex; and
incubating the separated target with one or more second materials that specifically bind a subpopulation of the target to separate the target subpopulation which is specifically bound to the one or more second materials.

19. The method according to claim 18, wherein the first and second materials specifically bind to different sites of the target.

20. The method according to claim 18, further comprising detecting the separated target subpopulation.
